(19) United States
(12) Patent Application Publication
(10) Pub. No.: US 2024/0207601 A1
(43) Pub. Date: Jun. 27, 2024

(54) CLAMPING ELEMENT FOR A PUMP DEVICE

(71) Applicant: Fresenius Medical Care Deutschland GmbH, Bad Homburg (DE)

(72) Inventor: Udo Kirstgen, Schweinfurt (DE)

(21) Appl. No.: 18/293,491

(22) PCT Filed: Jun. 30, 2022
(86) PCT No.: PCT/EP2022/068169

§ 371 Date: Jan. 30, 2024

(30) Foreign Application Priority Data
Jun. 30, 2021 (DE) 10 2021 116 833.3

(51) Int. Cl. A61M 60/835; A61M 60/113; A61M 60/279; A61M 60/37; A61M 60/849; F04B 43/12; F04B 53/16

(52) U.S. Cl. CPC A61M 60/835; A61M 60/113; A61M 60/279; A61M 60/37; A61M 60/849; F04B 43/1253; F04B 53/16

(57) ABSTRACT

The invention relates to a clamping element for securing a tube, having the following: a first guide and a second guide for receiving a tube, wherein the two guides run towards each other, and the clamping element is designed in such a manner that it is connectable to a pump device such that a tube guide can be formed by the two guides at a pump inlet and pump outlet.

### TECHNICAL FIELD

The invention relates to a clamping element for securing a tube according to the preamble of claim 1, to a pump device for a medical device according to the preamble of claim 10, to a blood treatment device according to the preamble of claim 15, and to a tube set according to the preamble of claim 16.

### BACKGROUND

Hose pumps, also called peristaltic pumps, are known from the prior art. This type of pump is frequently used in the medical sector for conveying fluids. In particular, hose pumps are used in dialysis machines, and therefore the conveyed fluid is, for example, blood, dialysis fluid, a drug solution for introducing drugs into the patient's blood, for example heparin, citric solution, iron complex solutions or other drugs, or else a substituate. A fluid guided in a tube is conveyed here, for example, by occlusion rollers attached to a rotor. The tube is placed here between the rotor and a stator. The fluid is conveyed in the circumferential direction by the occlusion rollers, wherein, in the conveyed fluid, positive pressure prevails in a conveying direction downstream of an occlusion roller and negative pressure prevails upstream of an occlusion roller. A hose pump typically has 2 or 4 of these occlusion rollers arranged radially on the rotor. Said pressures prevailing in a hose pump result in a loading both on the fluid being conveyed, but also on the tube used. In particular, excessive loadings may occur on the tube in the inlet or outlet region of the pump.

Since a change of a rotor or stator of a hose pump involves a high structural outlay, and the service life of medical devices is lengthy because of licence requirements, it is desirable to avoid changes of rotor or stator.

Similarly, reinforcement elements on the tube itself can be undertaken only to a very limited extent. Medical hoses for use in the case of dialysis machines have to satisfy particularly high hygienic standards and safety standards. In addition, tubes as disposables are subject to a high-cost pressure.

It is an object of the present invention to reduce the loading on the conveyed fluid and on the tube used.

### SUMMARY OF THE INVENTION

The object according to the invention is achieved by a clamping element with the features of claim 1, a pump device with the features of claim 10, and a blood treatment device with the features of claim 15.

The clamping element for securing a tube has a first guide and a second guide for receiving a tube. The two guides run towards each other here, and the clamping element is designed in such a manner that it is connectable to a pump device such that a tube guide can be formed by the two guides at a pump inlet and pump outlet.

By means of the arrangement of the guides which are preferably spaced apart further from each other in a direction towards the rotor than in a direction further away from the rotor, a tube guide is preferably achieved, in which the tube is inserted at an angle of less than 120°, preferably less than 90°, preferably less than 45° with respect to a tangent to the rotor. The tangent can be formed here, for example, at the insertion point of the tube on the rotor. For example, the tangent can be formed at the point at which the tube is guided for the first time along the circumference of the rotor.

In a preferred refinement, the clamping element can be designed here so as to be connectable to a pump device in such a manner that a tube guide at least at the pump inlet and/or pump outlet runs substantially tangentially with respect to a rotor of the pump device. The tube is thereby supplied tangentially with respect to the rotor of the pump device. The action of force of the rotor on the tube thus does not increase abruptly, but rather continuously. In addition, the tangential supply enables severe shearing forces in the tube itself to be prevented.

The tube to be used is in particular a tube for medical uses, in particular for a dialysis treatment, for example a tube for extra corporal circulation for guiding blood. Tubes provided for medical uses have to satisfy particularly high hygienic standards. These tubes are frequently composed of a PVC material and preferably do not have a coating here. Coatings could become detached during a treatment and cause the risk of particles entering the circulation. The reinforcing elements which can be applied to the tube are thereby restricted. The tangential supply of the tube enables high loadings on the tube and on the guided fluid, for example blood, to be prevented by structural means.

The two guides can be designed here as depressions, for example groove-shaped with a constant cross section or changing cross section, as protrusions, for example with lateral delimitations for positioning the tube or as geometrical receptacles or delimitations for positioning the tube on the clamping element. The guides can run here over the entire surface of the clamping element or only over a partial region of the surface. The guides can also have a radius which corresponds to the outer radius of the insertable tube. Similarly, however, the radius can also be greater or smaller or can change over the course of the guides. The guides can likewise be designed in such a manner that an insertable tube is received only at at least two points, that is to say in a punctiform manner, and the guides can also be designed in such a manner that the tube is received linearly or extensively.

The shape of the guides in a top view of the clamping element can also be virtually triangular. The position of an insertable tube with respect to the clamping element is determined by the guides. The shape of the guides here can be identical or different. The guides on the clamping element are designed here in such a manner that, when the clamping element is inserted into a pump device, a tube guide is produced at least at the pump inlet or at the pump outlet, by means of which the tube is supplied to, or guided away from, the pump device in a defined position. The complete securing of the tube can be brought about here entirely by the clamping element or in an interaction of clamping element and pump device.

The two guides can furthermore be designed in relation to each other in such a manner that, in the event of an imaginary extension of the guides, an angle between them of 5° to 90°, preferably of 10° to 60°, even more preferably of 25° to 50°, is formed.

According to one development, the clamping element can be at least partially elastic, in particular a first side portion of the clamping element can be elastic.

An at least partially elastic design of the clamping element facilitates a connection of the clamping element to the pump device. The clamping element here can be elastic on at least one side in such a manner that the clamping element can easily be inserted into and removed from the pump device. The clamping element can therefore be formed from an elastic material, for example an elastic plastic. In other words, a thermoplastic elastomer and silicone or rubber can be used.

According to one development, the clamping element can have at least one connecting element for connecting to a pump device, in particular a protrusion, preferably a pin-shaped protrusion. The connecting element can be formed here on a second side portion which lies opposite the first side portion.

The connecting element can be designed in such a manner that it permits a releasable connection of the clamping element to the pump device. In other words, the connecting element is an element formed geometrically on the clamping element in such a manner that, when the clamping element is connected to the pump device, at least the position in one dimension between clamping element and pump device is unambiguously defined. In other words, the connecting element can be a slot-shaped recess on the clamping element or a spring-shape protrusion.

The connecting element can also be designed as a pin-shaped protrusion or a pin-shaped recess on the clamping element. The connecting element is preferably formed on a second side portion which lies opposite the first side portion. The installation of the clamping element is thereby further simplified. In particular, firstly the clamping element can thereby be firstly inserted with the second side portion and a connection by means of the connecting element achieved such that complete insertion of the clamping element is easily possible by means of the elastic first side portion.

According to one development, the two guides can be formed on a first portion, wherein the first portion can preferably be rotatable about an axis of rotation of a second portion.

In other words, the clamping element can have at least two portions which are movable with respect to one another. An axis of rotation can be formed or mounted here on the second portion. The first portion, on which the guides are formed, can be connected here to the second portion so as to be rotatable about said axis of rotation. The axis of rotation can be formed here integrally with the second portion. For example, the axis of rotation can have two pin-shaped protrusions to which the first portion is connectable, for example attachable.

Alternatively, an axis of rotation about which the first portion is rotatable can be mounted in the second portion. By means of such a connection, the position between the first portion and the second portion can be changeable. In particular, the first portion can be arranged tiltable on the second portion in such a manner that the first portion changes the position of the guides relative to the second portion and thus relative to the pump device.

According to one development, the first portion can be preloaded, preferably by means of a spring, in particular a spiral spring, in a first position, wherein, in the first position, the first portion is at least partially spaced apart from the second portion.

According to one development, the axis of rotation of the first portion can be arranged on the second portion.

According to one development, the axis of rotation can be mounted on the second portion and the spiral spring can be mounted on the axis of rotation.

In other words, the clamping element can have two portions arranged moveably with respect to each other. A spring element which preloads the first portion against the second portion can be formed here on the first and/or second portion. However, the spring element here can also be arranged in a manner connected to the axis of rotation arranged on the second portion. In particular, a spiral spring can be positioned on the axis of rotation, such that the first portion is arranged in a tilted manner against the second portion. Therefore, for example, a force acting on an inserted tube can be designed to be changeable. During the insertion of the tube, the first portion can thus be pressed in the direction of the second portion. Tubes of different diameters can thereby likewise be held securely by the distance between the first and second portion being changeable.

According to one development, the first and/or second guide can be groove-shaped. Similarly or alternatively, the first guide and/or the second guide can receive the tube on two opposite sides of the tube.

In other words, at least one guide can be groove-shaped, wherein said guide in cross section spans a circular sector of less than 180°, in particular a circular sector of less than 90°, in particular a circular sector of less than 45°, in particular a circular sector of less than 30°. Similarly, at least one guide can be groove-shaped, wherein said guide in cross section spans a circular sector of 180° or greater. Alternatively, at least one guide can have two opposite side walls and can be virtually U-shaped. Alternatively, at least one of the guides can have an omega shape, such that the tube can be pressed into the guide.

According to one development, the clamping element can be assigned an extension. Said extension can preferably extend above the first guide and/or the second guide such that a tube can be clamped between first guide and extension and/or can be clamped between the second guide and extension. The extension here can be formed integrally with the clamping element. Similarly, the extension can be assigned to a pump device or a pump housing of a pump device and can interact with a clamping element brought into connection with the pump device. By means of the extension, the tube can be fixed between clamping element and extension by static friction. This prevents displacement of the tube after the latter has been inserted. For example, the clamping element can be configured to be L-shaped.

In other words, the extension enables the tube to be held on a further portion of the extension formed on the circumference of the tube. By this means, the reception of the tube is improved. In particular, the extension can be formed at a point lying opposite a guide, such that the tube can be clamped. The extension can be formed here both on the first and on the second portion of the clamping element. The extension is preferably formed on the second portion. If the first portion is movable or preloaded with respect to the second portion, the distance between extension and first portion can be changed, or the tube can be clamped between extension and first portion by means of the spring force.

Furthermore, a pump device can be provided for a medical device, the pump device having a clamping element according to one of the preceding aspects.

According to one development, the pump device furthermore has a pump housing with a pump bed, a rotor which is formed in the pump bed and on which at least one occlusion element is arranged, wherein a tube can be placed between pump bed and rotor in such a manner that it is pressed by the occlusion element radially against the pump bed such that, when the rotor rotates, fluid can be transported in the tube, a pump inlet which is formed in the pump housing and in which the tube is supplied to the pump device, a pump outlet which is formed in the pump bed and in which the tube is guided out of the pump device.

According to one development, the clamping element can be insertable into the pump housing in such a manner that the clamping element forms part of a tube guide and the tube can be secured at the pump inlet and at the pump outlet by the clamping element.

In other words, the clamping element can form a partial portion of the tube guide at the pump inlet and pump outlet. Part of the tube guide here can be formed by the clamping element and part of the tube guide can be formed at the pump inlet and pump outlet of the pump housing. Alternatively, the tube guide can be defined at the pump inlet and pump outlet only by means of the clamping element. For example, the pump housing at the pump inlet and/or pump outlet can have protrusions which form a partial portion of the pump guide. A defined tube guide can thus be achieved by this interaction between the pump housing and the clamping element.

According to one development, the tube guide at the pump inlet and/or pump outlet takes place, preferably by the clamping element, in such a manner that the first guide and the second guide run towards each other in such a manner that the first guide and the second guide are spaced apart further in the direction of the rotor. In other words, the guides can be spaced apart further on the clamping element on a portion which lies closer to the pump device, for example the rotor, than on a portion which lies further away from the pump device, for example the rotor. Similarly or alternatively, the tube guide at the pump inlet and/or pump outlet can take place, preferably by the clamping element, in such a manner that the tube can be supplied tangentially with respect to the rotor.

According to one development of the pump device for a medical device, the pump inlet and the pump outlet can be formed by the pump housing and clamping element in such a manner that the tube can be secured at the pump inlet and/or at the pump outlet on in each case opposite sides.

Furthermore, a blood treatment device with a pump device according to preceding aspects, and a clamping device according to preceding aspects can be provided.

The blood treatment device can be in the form of a dialysis device, haemodialysis device, haemofiltration device, haemodiafiltration device, apheresis device, or plasma treatment device.

Similarly, a tube set, preferably for extracorporal blood treatment, preferably dialysis treatment, can be provided. A partial portion of a tube for extracorporal blood treatment, preferably dialysis treatment, can be connected here to a clamping element according to the invention. The tube can be connected here to the first guide and/or the second guide prior to the insertion into a blood treatment device. If the tube is not connected to the two guides, the tube can be connected to the other guide after insertion of the clamping element into a blood treatment device.

The clamping element according to the invention, pump device or blood treatment device thus makes it possible to reduce the forces acting on the tube and on the medium guided in the tube. In particular, smaller forces occur because of the tube entering the pump device obliquely or tangentially with respect to the rotor of the pump device. This is the case in particular since an action of force of the pump device on the tube does not increase abruptly.

Since the tube is already inserted tangentially, or in the circumferential direction of the rotor, that is to say along the rotational movement, the tube does not undergo any abrupt change in direction. On the contrary, the tube is already inserted in the circumferential direction such that the force acting on the tube gradually increases. In other words, striking of an occlusion element on the tube with a continuously increasing action of force is provided. The avoidance of an abrupt action of force also has a positive effect on the fluid guided in the tube. If, for example, blood is guided in the tube and an abrupt action of force occurs, this may lead to blood damage. By means of the teaching according to the invention, an abrupt action of force and thus damage to the fluid guided in the tube and to the tube itself can be avoided.

In addition, it is made possible by means of the clamping element according to the invention to permit a change in the entry or exit geometry without this geometry having to be undertaken at the pump housing itself. This provides a cost-effective and structurally simple possibility of achieving a desired inlet or outlet geometry for the tube in the case of already existing pump devices.

### BRIEF DESCRIPTION OF THE DRAWINGS

The present invention will be explained by way of example below with reference to the attached drawings in which the same reference signs denote identical or similar components. In the figures of the drawing:

FIG. 1 shows a schematic illustration of a pump device;

FIG. 2 shows a simplified flow pattern of a fluid system of a blood treatment device;

FIG. 3 shows a schematic illustration of a partial cutout of a front view of a blood treatment device;

FIG. 4 shows a schematic illustration of the clamping element in a side view;

FIG. 5 shows a schematic illustration of a clamping element in a top view.

### DETAILED DESCRIPTION OF AN EXEMPLARY EMBODIMENT

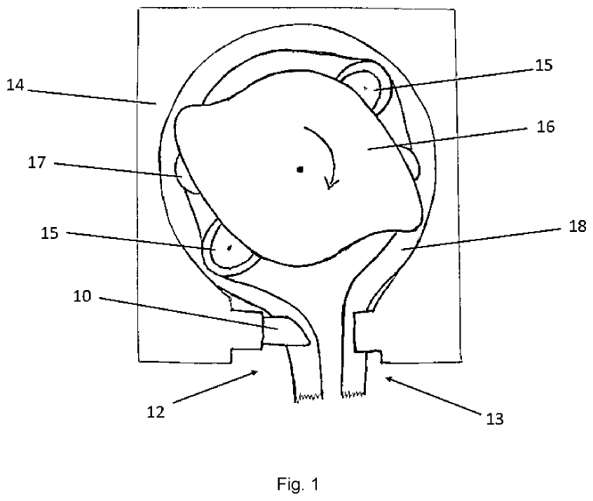

The pump device shown in FIG. 1 has a pump housing 14. The rotor 16 and the occlusion elements 15 and guide elements 17 are arranged in the pump housing 14. The pump housing 14 also has a pump bed. A tube 18, as shown in FIG. 1, can be inserted into said pump bed. The tube 18 can be a blood tube, a dialysis fluid tube, or a substituate tube.

Analogously to this, the pump device can also be a hose pump or else a peristaltic pump and can be used as a blood pump, a dialysis fluid pump or a substituate pump. Such pumps can be used in a blood treatment device 20.

FIG. 1 additionally also shows axial guide elements 17 on the circumference of the rotor 16. The guide elements 17 serve for aligning the tube and in particular prevent the tube from slipping out of the tube bed. The axial guide elements are preferably attached rotatably here such that no friction arises between guide elements 17 and tube. As illustrated, two guide elements 17 can be attached to the rotor 16. Similarly, a plurality of guide elements 17, for example three or four guide elements, or else only one guide element 17 can be provided on the rotor 16.

A covering is provided on the front side of the rotor 16. The rotor can also have a handle, not illustrated here, which can be unfolded from the rotor 16. This handle also enables the rotor 16 to be rotated manually and the pump to carry out the pumping operation. In the pump device shown in FIG. 1, the clamping element 1 is already inserted into the pump device. From this perspective, only one extension 10 of the clamping element 1 can therefore be seen. This extension 10 serves for fastening the tube in a direction out of the plane. As can be seen in FIG. 1, the clamping element 1 enables the tube to be supplied tangentially with respect to the rotor 16. The occlusion element 15 therefore does not bring about an abrupt action of force on the tube. On the contrary, the action of force is increased slowly after the first contact between occlusion element 15 and tube. By this means, blood damage that can be caused by an abrupt increase in pressure in the tube can be avoided. The tangential insertion of the tube also makes it possible to avoid shearing forces caused by a forced sharp change in direction due to the occlusion element striking against the tube.

In addition, FIG. 1 shows occlusion elements 15 attached to the rotor 16. These occlusion elements 15 are formed cylindrically here in the form of rollers. By way of example, two occlusion elements 15 are formed on the circumference of the rotor. Similarly, however, three or four occlusion elements or only one occlusion element can also be provided. The occlusion elements 15 are designed in such a manner that they press the tube radially outwards against the pump bed. For this purpose, the occlusion elements can be preloaded by means of a spring. By means of the occlusion elements 15 pressed against the tube and by means of the rotation of the rotor 16, fluid is thus conveyed in the tube.

If a tube is inserted into the pump device and the pump device rotates in the clockwise direction, as shown in FIG. 1, the fluid is thus conveyed in the tube from the pump inlet 12 to the pump outlet 13. In the case of a blood pump, blood can thus be drawn from the patient and supplied via the arterial line to a dialyser 21 by means of the blood pump. The dialyser 21 has a blood chamber, which is connected to the venous and arterial blood line, and a dialysis fluid chamber. These chambers are separated from each other by a semi-permeable membrane. In the dialyser 21 itself, the blood flows on the counterflow principle, with dialysis fluid flowing on the other side. The blood side of the dialyser 21 and the blood pump are part of the extracorporal blood circulation.

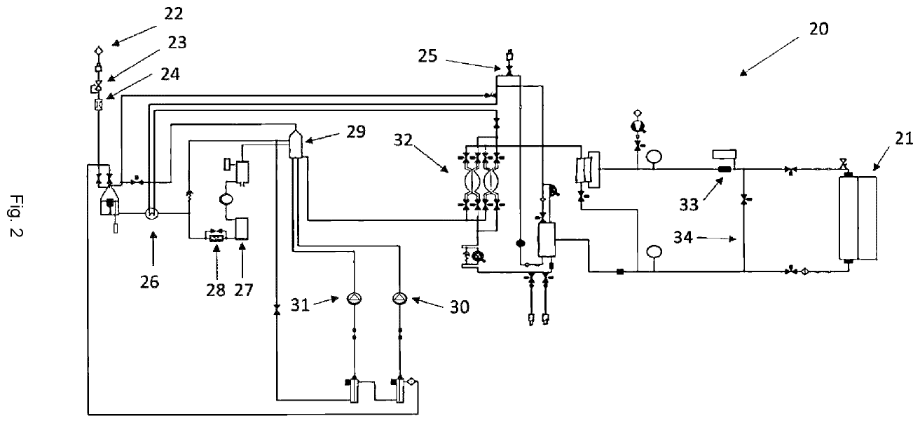

FIG. 2 shows a simplified flow diagram of a fluid system of a blood treatment device 20 as a dialysis machine. The fluid is supplied to the blood treatment device 20 via a dialysis water connection 22, a downstream pressure-reducing valve 23, which reduces the pressure, for example, to approximately 0.5 bar, and an inlet throttle 24. Permeate, that is to say softened and filtered water, is supplied via the dialysis water connection 1.

After passing through the dialyser 21, the dialysate is supplied via a dialysis fluid removal line and a drain valve 25 to a drain. The fresh dialysis fluid can be heated here by the dialysate via a heat exchanger 26 in order subsequently to be heated further, for example by means of a heating coil or a heating bar. In addition, the permeate is subjected to degasification in a degasification chamber 27. In order to promote removal of air from the permeate, the permeate is exposed for this purpose to a negative pressure by means of a degasification throttle 28. By means of the temperature increase and pressure reduction, air can therefore escape in the form of bubbles via a downstream air separator 29.

After the degasification of the permeate, the mixing fluid, here the dialysis fluid, is produced by admixing at least one concentrate solution. In order to provide the fresh dialysis fluid, permeate is thus supplied via the dialysis water connection 22, and, for example, two concentrate solutions, for example a bicarbonate concentrate solution and an acid concentrate solution, are supplied, for example from concentrate containers, not illustrated here, and subsequently mixed. The concentrate solutions are conveyed here via concentrate pumps 30, 31. The concentrate pumps 30, 31 can be designed, for example, as reciprocating pumps, diaphragm pumps or geared pumps. The proportioning, that is to say the mixture of acid concentrate and bicarbonate with permeate in a predetermined ratio, can take place volumetrically or in a conductivity-controlled manner. In the case of the volumetric proportioning shown in this exemplary embodiment, the supplied volume is achieved via a clocked supply by means of the concentrate pumps 30, 31, for example reciprocating pumps.

The dialysis fluid produced by the mixing subsequently flows through part of a balance chamber 32 and thus enters the dialysis fluid circuit. The balance chamber 32 provides a balance here between the fresh dialysis fluid and the used dialysis fluid, the dialysate. A mixing fluid sensor 33 is connected upstream of the dialyser 21 in order to check the correct composition of the dialysis fluid. A bypass valve 34 is connected downstream of the mixing fluid sensor 33, for example in the form of a conductivity sensor. If the mixing fluid sensor 33 during the dialysis treatment detects a non-physiological fluid, that is to say a fluid which does not meet a predetermined condition, for example a predetermined conductivity, the bypass valve 34 is opened and the dialysis fluid conducted via a line portion 34.

The actuators, pumps and valves of the blood treatment device can be connected to, or can be in communication with, a control device, not illustrated here.

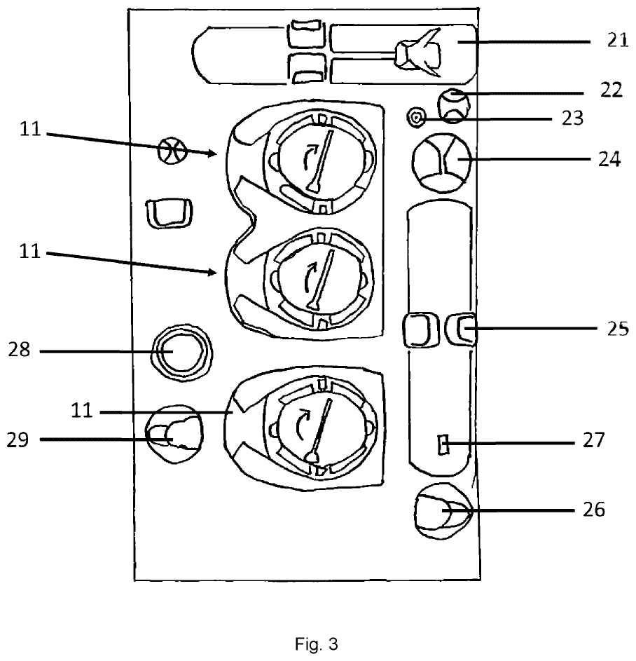

FIG. 3 shows a partial cutout of a front view of an extracorporal blood treatment device 20. The blood circulation here can have a tube system through which the patient's blood can flow during the treatment. The tube system can be inserted into a dialysis machine for the treatment and can interact with modules, for example blood modules of the dialysis device. The blood module here can have an arterial patient tube clamp 29 of an arterial portion of an arterial blood line. As described, the blood is extracted from a patient by means of an arterial connection needle and returned by means of a venous connection needle. In a corresponding manner, the blood module can likewise have a venous patient tube clamp 26, wherein the blood downstream of said patient tube clamp 26 is supplied again to the patient via the venous connection needle.

The partial cutout of the blood treatment device 20 that is shown in FIG. 3 has three hose pumps 11 from top to bottom, wherein the first pump is a single needle pump, the second pump is a blood pump, and the third pump is a substituate pump. An extracorporal blood treatment device here can have all three hose pumps, but also only a blood pump or only a blood pump and a substituate pump. Substituate can be supplied by pre-dilution or post-dilution to the arterial or venous blood line by means of the substituate pump.

In addition, a feed point for heparin or another anticoagulant can be provided. The blood treatment device can thus have a heparin pump 21, an arterial pressure-measuring unit 28 and sensors 25, 27. Said sensors 25, 27 monitor the tube for air bubbles and for the contents. An ultrasonic sensor and an optical sensor for identifying colour can thus be provided. Finally, the blood treatment device has an arterial pressure-measuring unit 28 and a venous pressure connection 23.

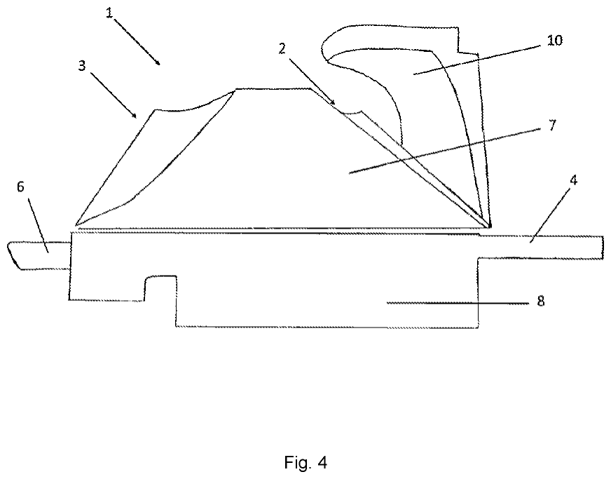

FIG. 4 shows a clamping element 1 according to the invention in a side view. The clamping element can be connected to one of the previously described hose pumps 11. In particular, the tube guide can be formed at the pump inlet 12 and pump outlet 13 by insertion of the clamping element 1 into the pump housing 14. For this purpose the clamping element 1 is connected to the pump housing 14 via a connecting element 6, a protrusion as illustrated in FIG. 3. A corresponding depression or slot can be formed for this purpose in the pump housing 14. Alternatively, a protrusion can be formed on the pump housing 14 and a corresponding depression or slot on the clamping element 1 as the connecting element 6. In other words, a form-fitting connection can be formed between clamping element 1 and pump housing 14. A magnetic connection can also be formed between clamping element 1 and pump housing 14.

For simple installation, the clamping element 1 is firstly inserted with the second side portion 5, on which the connecting element 6 is formed, obliquely into the pump housing 14. The clamping element 1 can subsequently be completely inserted by simple pushing in. This is possible in particular since the second side portion 4 can have an elastic element. For this purpose, as illustrated in FIG. 3, an elastic side plate can be formed on the second side portion 4. Said side plate can be formed in particular from an elastic plastic. In other words, a thermoplastic elastomer or silicone or rubber can be used.

For removal of the clamping element 1 from the pump housing 14, the clamping element can be correspondingly grasped via an extension 10 and pushed in the direction of the second side portion 5. Subsequently, the clamping element 1 with the first side portion 5 of the elastic element can firstly be raised and then completely removed from the pump housing 14.

The extension 10 formed on the clamping element 1 is formed in particular above the first guide 2. Additionally or alternatively, an extension can also be formed above the second guide 3. The extension 10 can extend from a first portion 7 or second portion 8 of the clamping element 1. The extension 10 preferably extends from the second portion 8 which, in the installed state of the clamping element 1, is irremovable relative to the pump housing 14. The extension 10 is suitable in particular for securing a tube on at least two sides.

The extension can secure the tube from an upper side and the first guide 2 or second guide 3 can secure the tube from a lower side. The tube can be inserted from one side, from the left side with respect to the exemplary embodiment shown in FIG. 3, and can be held by an upper side, that is to say by a direction leading away from the pump housing 14. Similarly or alternatively, the tube can be secured by three points or extensively. The extension 10 can have an arcuate form here on a lower side which points towards the first guide 2 or second guide 3. The radius of the arcuate form can correspond here to the radius of a conventional tube. In particular, the radius can have 5 mm to 30 mm, in particular 10 mm to 20 mm.

The first guide 2 and/or the second guide 3 can be semicircular or greater than 180° in cross section. The first guide 2 and/or the second guide 3 can be in the form, for example, of only a quarter circle or more than a quarter circle. In particular, the first guide 2 and/or the second guide 3 can be formed with an arcuate form of 20° to 270°, preferably 30° to 180°, even more preferably of 35° to 90°, even more preferably approximately 45°.

The clamping element 1 shown in FIG. 4 can be connected to a tube after insertion into the pump housing 14. In particular, after the insertion, the tube can be inserted into the first guide 2 and the second guide 3. Alternatively, a tube, not illustrated in

FIG. 4, can already be connected to the clamping element 1 prior to the insertion. The clamping element here can be part of a tube set. The tube together with the clamping element can be designed in this case as a disposable.

For example, the tube can be connected in the manner of a loop to the first guide 2 and the second guide 3 already prior to the insertion into a pump housing 14. Alternatively, the tube can also be connected only to one of the guides 2, 3 prior to the insertion and, after the insertion into the pump housing 14, can be connected to the other guide 2, 3. If the tube is already connected to the clamping element 1 prior to the insertion, the handling is thereby facilitated. In particular, the handling steps are reduced by the clamping element 1 with the tube already inserted prior to the insertion. This is particularly advantageous since the time for setting up a dialysis device must take up only a certain time so as not to put the strict planning of time at risk.

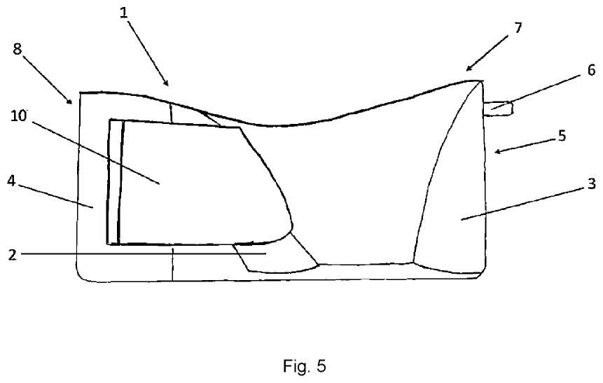

FIG. 5 shows the clamping element 1 schematically in a top view. The shape of the guides 2, 3 here projected onto a plane is virtually triangular. In the embodiment shown here, the protrusion 6, for connecting the clamping element 1 to the pump device, is formed at the edge of the clamping element 1. Alternatively, however, the protrusion 6 can also be formed centrally or a plurality of protrusions 6 can be formed at various points.

The clamping element 1 can furthermore have an axis of rotation, not illustrated here. The axis of rotation can be formed here in the second portion 8. The second portion 8 can also have a spring, not illustrated here. Said spring can be mounted as a spiral spring on the axis of rotation. The first portion 7 can be connected to the second portion 8 so as to be rotatable about said axis of rotation. A tube inserted into the guides 2, 3 can thus be preloaded against the extension 10 or elements of the pump housing.

1. Clamping element (1) for securing a tube, having
a first guide (2) and a second guide (3) for receiving a tube,
wherein the two guides (2, 3) run towards each other, and the clamping element (1) is designed in such a manner that it is connectable to a pump device (11) such that a tube guide can be formed by the two guides at a pump inlet (12) and a pump outlet (13) of the pump device (11).

2. Clamping element according to claim 1, wherein the clamping element (1) is at least partially elastic, in particular a first side portion (4) of the clamping element (1) is elastic.

3. Clamping element according to claim 1 or 2, wherein the clamping element (1) has at least one connecting element (6) for connecting to a pump device, in particular a protrusion, in particular a pin-shaped protrusion, wherein said connecting element (6) is preferably formed on a second side portion (5) which lies opposite the first side portion (4).

4. Clamping element according to claims 1 to 3, wherein the two guides (2, 3) are formed on a first portion (7), wherein the first portion (7) is preferably rotatable about an axis of rotation of a second portion (8).

5. Clamping element according to claim 4, wherein the first portion (7) is preloaded, preferably by means of a spring, in particular a spiral spring, in a first position, wherein, in the first position, the first portion (7) is at least partially spaced apart from the second portion (8).

6. Clamping element for securing a tube according to claim 4 and/or 5, wherein an axis of rotation about which the first portion (7) is rotatable is arranged on the second portion (8).

7. Clamping element for securing a tube according to claim 6, wherein the axis of rotation is mounted on the second portion (8) and the spiral spring is mounted on the axis of rotation.

8. Clamping element for securing a tube according to at least one of claims 1 to 7, wherein the first and/or second guide (2, 3) is groove-shaped, and/or wherein the first guide (2) and/or the second guide (3) receives the tube on two opposite sides of the tube.

9. Clamping element for securing a tube according to at least one of claims 1 to 8, wherein the clamping element is assigned at least one extension (10) which extends above the first guide (2) and/or the second guide (3) such that a tube can be clamped between first guide (2) and extension (10) and/or second guide (3) and extension (10).

10. Pump device for a medical device, having
a clamping element (1) according to at least one of claims 1 to 9.

11. Pump device according to claim 10, furthermore having
a pump housing (14) with a pump bed (12),
a rotor (16) which is arranged in the pump bed (12) and on which at least one occlusion element (15) is arranged,
wherein a tube can be placed between pump bed (12) and rotor (16) in such a manner that it is pressed by the occlusion element (15) radially against the pump bed (12) such that, when the rotor (16) rotates, fluid can be transported in the tube,
a pump inlet (12) which is formed in the pump housing (14), and in which the tube is supplied to the pump device, and
a pump outlet (13) which is formed in the pump bed, and in which the tube is guided out of the pump device.

12. Pump device according to claim 10 or 11, wherein the clamping element (1) can be inserted into the pump housing (14) in such a manner that the clamping element (1) forms part of a tube guide, and the tube can be secured at the pump inlet (12) and at the pump outlet (13) by the clamping element (1).

13. Pump device according to at least one of claims 10 to 12, wherein the tube guide at the pump inlet (12) and/or pump outlet (13) takes place, preferably by the clamping element, in such a manner that the first guide (2) and the second guide (3) run towards each other in such a manner that the first guide (2) and the second guide (3) are spaced apart further in the direction of the rotor (16), and therefore a tube can preferably be supplied tangentially with respect to the rotor (16).

14. Pump device according to at least one of claims 10 to 13, wherein the pump inlet (12) and the pump outlet (13) are formed by the pump housing (14) and the clamping element (1) in such a manner that a tube can be secured at the pump inlet (12) and/or at the pump outlet (13) on at least two tube sides, in particular on at least two opposite tube sides.

15. Blood treatment device with a pump device according to at least one of claims 10 to 14, and with a clamping device according to at least one of claims 1 to 9.

16. Tube set with a clamping element according to one of claims 1 to 9, preferably for use in a pump device according to one of claims 10 to 14.
